(19) United States
(12) Patent Application Publication
(10) Pub. No.: US 2021/0334923 A1
(43) Pub. Date: Oct. 28, 2021

(54) RAPID DISASTER RESPONSE MANAGEMENT SYSTEM

(71) Applicant: iParametrics, LLC, ALPHARETTA, GA (US)

(72) Inventors: ANDREW J. KLEIN, CANTON, GA (US); JOHN M. LORIMER, ALPHARETTA, GA (US); PAUL S. PELLETIER, JR., ALPHARETTA, GA (US)

(21) Appl. No.: 17/243,482

(22) Filed: Apr. 28, 2021

Related U.S. Application Data
(60) Provisional application No. 63/016,729, filed on Apr. 28, 2020.

(51) Int. Cl. G06Q 50/26; G06Q 40/08; G06Q 10/06; G06F 9/451; G06N 20/00

(52) U.S. Cl. CPC G06Q 50/26; G06Q 40/08; G06Q 10/06393; G16H 50/80; G06F 9/451; G06N 20/00; G06Q 10/06375

(57) ABSTRACT

Disclosed are various embodiments for a rapid disaster response management system. In one embodiment, data is received indicating a plurality of event drivers for a first plurality of geographic areas respecting one or more historical events. Data is received indicating whether individual ones of the plurality of geographic areas were assigned first disaster designations for the one or more historical events. A machine learning model is trained to determine correlations between the plurality of event drivers and the first disaster designations.

### CROSS-REFERENCE TO RELATED APPLICATIONS

This application claims the benefit of U.S. Provisional Application No. 63/016,729, entitled “RAPID DISASTER RESPONSE MANAGEMENT SYSTEM,” and filed on Apr. 28, 2020, which is incorporated herein by reference in its entirety.

### BACKGROUND

Currently whenever a disaster such as a hurricane occurs, the disaster sets in motion a sequence of responses designed to minimize the destruction and effect an eventual full recovery. After the initial Emergency Management phase, the Disaster Recovery phase begins. The goal of the Disaster Recovery phase is to restore communities as quickly as possible. The first part of this phase involves a high-level evaluation of the extent of damage so that federal funds can be mobilized for rebuilding, if appropriate. The Disaster Recovery phase also involves the process of securing resources, with the correct expertise and mobilizing them to the correct locations. However, currently, the process of generating the initial damage assessments required to apply for and obtain approval for funding as a federal disaster under the Federal Emergency Management Agency (FEMA) can take 40 days or longer. This is time wasted, before any significant recovery action can begin.

Specifically, when a disaster strikes, a determination must be made to measure if the resources needed to recover are expected to exceed local and state capability—if needed resources do exceed state and local capabilities, then a Presidential Disaster Declaration is made and only then can FEMA Public Assistance operations begin. This determination is made by performing Preliminary Damage Assessments often called (PDAs). PDAs are very resource and time-intensive, typically taking weeks to complete, and because they are rough windshield estimates, their accuracy is commonly off by orders of magnitude.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1-21 depict examples of user interfaces generated according to various embodiments of the present disclosure.

FIG. 22 is a schematic block diagram of a networked environment according to various embodiments of the present disclosure.

FIG. 23 is a flowchart illustrating one example of functionality implemented as portions of an interactive application and/or an analysis application executed in a computing environment in the networked environment of FIG. 22 according to various embodiments of the present disclosure.

### DETAILED DESCRIPTION

The Rapid Disaster Response Management System in various embodiments is designed to greatly expedite post-disaster Public Assistance response by identifying the specific locations (e.g., counties, ZIP codes, census tracts, etc.) of major impact, the scope of impact, and the characteristics of those locations in order to facilitate planning and mobilization of the appropriate funding and resources. This system is designed to augment the FEMA Preliminary Damage Assessment process by providing an immediate picture of the locations likely to be declared eligible for Federal disaster assistance within a short time period (e.g., a day or two) after a disaster, rather than in weeks. The Rapid Disaster Response Management System can function for a variety of disasters, including, but not limited to, hurricanes, tropical storms, severe thunderstorms, tornadoes, blizzards, epidemics, droughts, forest fires, criminal activity, and so on.

The Rapid Disaster Response Management System in various embodiments may identify immediately the majority of the locations likely to be declared Federal disaster areas and therefore eligible for Public Assistance grants, identify the overall scope of likely Federal assistance, identify generally the range of damage cost to be expected for each of the projected declared disaster locations, and identify immediately the characteristics of areas affected so that proper resources can be mobilized quickly (e.g., number of school children impacted, number of hospitals, amount of vegetative cover related to potential for debris, etc.). In various embodiments, the system will return such information down to the physical location of each of the facilities in the county or other area. This expedites early identification and mobilization of both personnel resources and supplies. Further, the system may identify critical infrastructure and at-risk populations potentially impacted so action can be taken pro-actively (e.g., public power stations, water treatment facilities, nursing homes, historical buildings, etc.). Also, the system may provide a basis for initiating bridge funding with confidence.

The Rapid Disaster Response Management System in various embodiments provides different levels of use. An overview dashboard user interface is used to gain an overview of the entire disaster. A state dashboard user interface may be used by state managers to gain a more-focused, state-specific overview of the disaster. Drill downs that are regional and/or topical from either the overview dashboard or the state dashboard can be used to break down the response and to allocate resources. An appended data summary user interface allows users to select which characteristics they wish to focus on, either by category or by characteristic and generate detailed operational reports. For example, the operational reports may be generated as spreadsheets or portable document format (PDF) files. For example, a report may be generated of the addresses for all of the hospitals in a given impacted county, together with data on number of beds and other details. These reports can be used to supply responders with actionable information.

Embodiments use a combination of machine learning and geospatial and visualization technology in order to project what locations are likely to be declared federal disaster areas within a few days of an event. In one embodiment, the system can currently project more than 57% of the counties that will eventually be declared eligible for Federal Disaster Assistance with a greater than 95% accuracy (meaning that if the system “declares” a county, there is only a 5% chance that it will not eventually be declared). The system can also project, on a county by county basis, the general range of expected damage cost. Currently, the damage is quantified into three broad cost ranges ($1,000-$100,000, $100,001-$10,000,000 and $10,000,001-$100,000,000), but other cost ranges can be used if desired.

In one implementation, this system ultimately brings together over 50 disparate data sets from many different data sources, both publicly available and private or restricted. Since these data sources are not generated specifically for the purpose of this system, making them work together requires a considerable amount of data analysis, data manipulation, cleaning, normalization and reformatting. The data sets utilized include both historical data sets and real-time or near-real-time data feeds. The system may include automated and manual processes for keeping all of the data sets continuously up-to-date and maintaining quality control. The result of this process is a unique data repository that has been specifically assembled to address the requirements of this problem.

The system can be driven by machine learning algorithms that are used to identify the likely declared counties, and the likely range of damage by county and FEMA category. The specific algorithms in use can be highly specific to, and tuned uniquely to, specific disaster types, such as hurricanes, forest fires, criminal activity, etc. The user interface can be optimized for use specifically by users involved day-by-day in disaster response activities. The user interface can designed to provide such users with operation information quickly and efficiently, with a minimal learning curve.

The Rapid Disaster Response Management System is comprised of two main modes: an analytics mode and an operational mode. The purpose of the operational mode is to provide disaster managers with a tool that they would use during an actual emergency to take practical actions. Field users of the tool do not need to understand the science behind the tool; they just need information that will help them to do the job at hand.

The analytics mode includes the data acquisition, data processing and data science pieces needed to provide the probabilities of declaration. The analytics mode may include data from many different data sources from various agencies that are either major drivers in determining whether or not a county will be declared or that will provide better situational awareness of the event's impact. For hurricane events, this included weather drivers, such as the hurricane wind swath and precipitation, as well as more static drivers, such as population, vegetative cover, and built density, and infrastructure data on critical facilities, such as hospitals, water treatment plants and electrical substations.

Various geographic information system (GIS), business intelligence (BI) and Data Science analytics applications can be used to process and transform the data into forms that are suitable for both the visualization and for machine learning. For example, GIS applications can be used to interpolate between rain gauges to get a continuous view of precipitation throughout the country for the machine learning model. Storm surge files can be processed in a raster format to understand the geographic extent and magnitude of storm surge on a county level and generated wind swath shapefiles based on wind radii values for historical hurricanes where wind swath shapefiles are not available.

Once the results of the model and visualization are refined and tested in the analytics mode, a separate application can be built from this framework as an operational tool. As an operational tool, the Rapid Disaster Response Management System is aimed at providing the user with a complete understanding of an approaching disaster, including the counties of major impact, the scope of impact and the characteristics of the impacted counties so that the user can begin to take appropriate actions as soon as possible. This tool aids in both preparedness and response by facilitating planning and mobilization of the appropriate resources and greatly expediting the Public Assistance response process, respectively. For example, an Emergency Manager may use the tool to quickly gauge the scope of recovery effort by identifying the majority of the counties likely to be impacted and the dynamics of those communities, while field personnel may use to the tool to generate detailed reports of the actual location of various facilities along with key metrics.

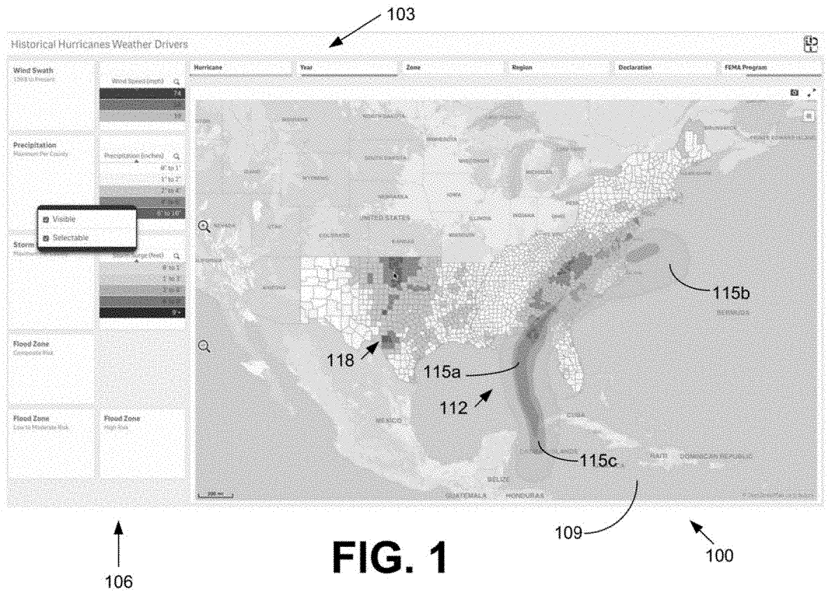

FIG. 1 shows an example view of a user interface 100 in analytics mode. FIG. 1 shows how the various data features that are drivers in whether or not a county will be declared a disaster after a hurricane event. The filter components 103 on the top of the sheet allows visualization of the data for a select hurricane—in this case Hurricane Michael from 2018. The user interface 100 shows the weather driver components 106, with the various data features are available for toggling on and off to visualize them spatially on the map 109. The wind swath 112 shows the wind swath of Hurricane Michael with a first portion 115a showing the 74 mph swath, the second portion 115b showing the 39 mph swath and the third portion 115c showing the 58 mph swath. The wind swath 112 can be overlaid on the precipitation layer 118 and other layers, with darker shading correlating to higher levels of precipitation, meaning these counties received more rain and do the same for the storm surge layer.

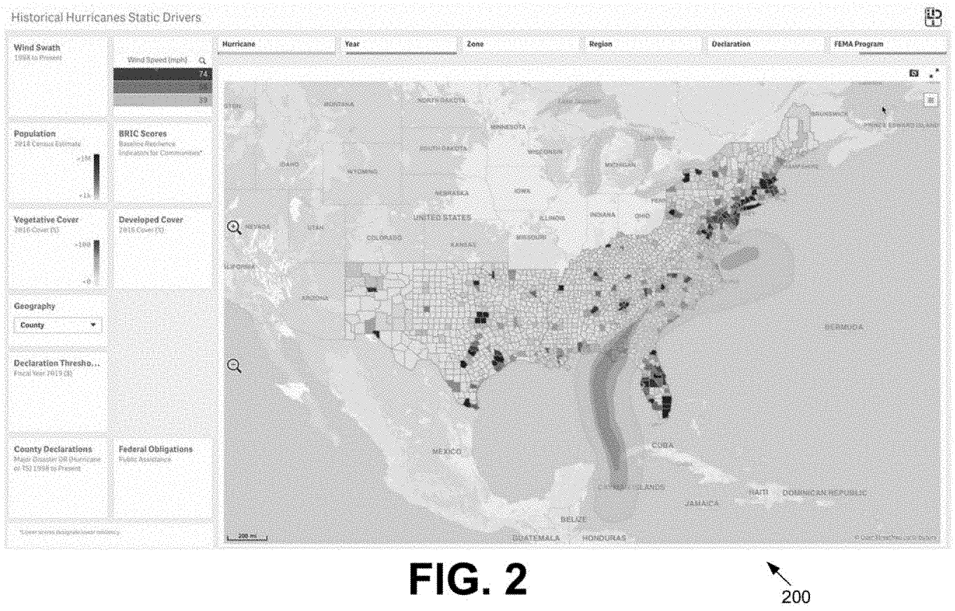

FIG. 2 shows a user interface 200 with more static drivers, or data features that are not likely to change very rapidly—such as, for example, features like population and vegetative cover. Different colors or patterns can be used for different drivers, with different shading of the pattern or color corresponding to intensity along a range.

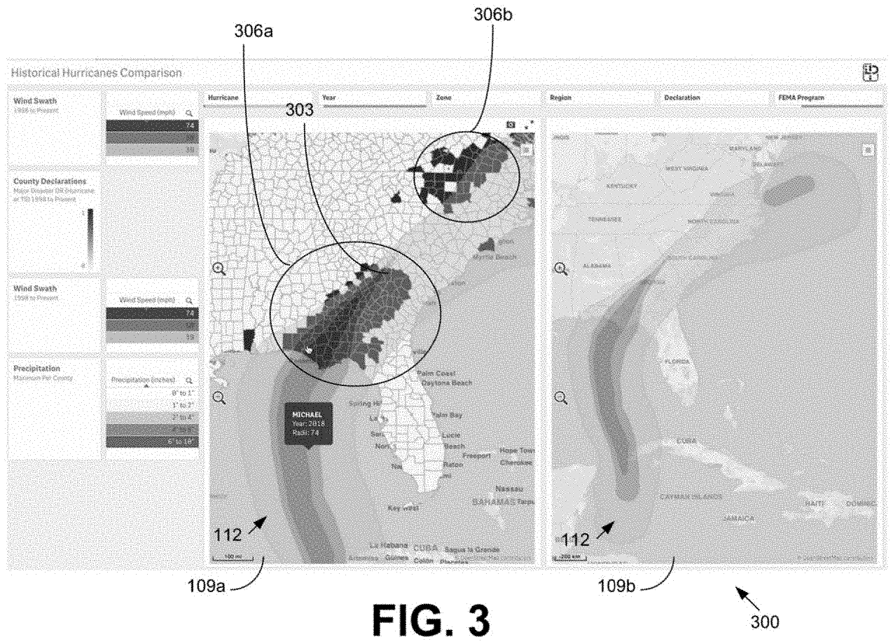

FIG. 3 shows a user interface 300 providing an analysis beginning with wind swath 112 alone as a major driver of the declaration process. The wind swath 112 of Michael relative to the disaster-declared counties 300 actually declared by FEMA is shown in dark shading and from this we can see that while wind swath 112 is probably a major driver, it cannot be the sole driver—we can come to this conclusion by zooming into the first map 109a—we can first see that majority of the counties 306a in Georgia, Florida, and Alabama that we declared fell within the wind swath 212, but if we go North, we can also see that a significant number of counties 306b in North Carolina and Virginia were declared yet do not fall within the wind swath 212. A second map 109b is shown with only the wind swath 112 at a different level of zooming.

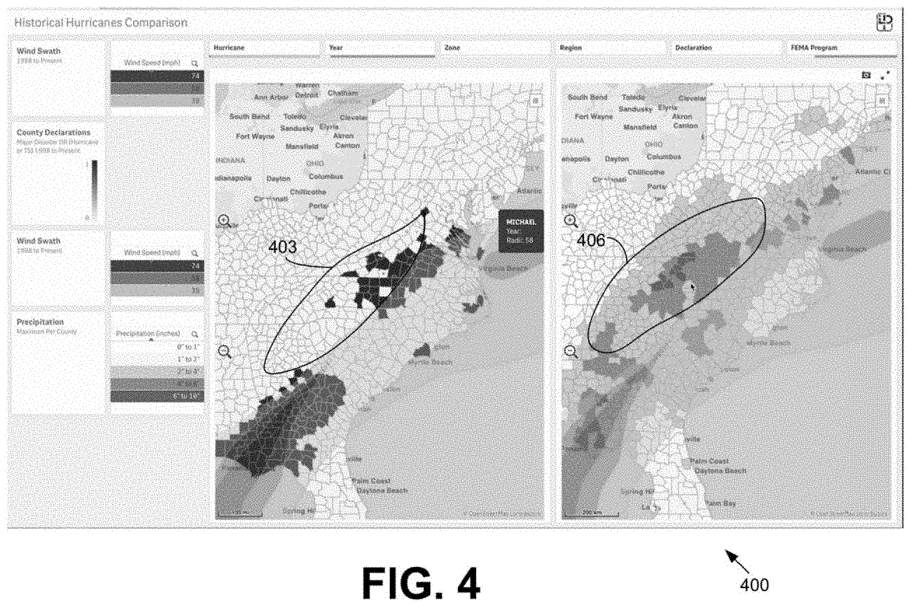

Looking at precipitation on the map on the right in the user interface 400 of FIG. 4, one can observe that a large number of counties that were declared but not in the wind swath, as shown at 403, experienced high levels of precipitation as shown at 406, leading to a conclusion that precipitation is also a major driver.

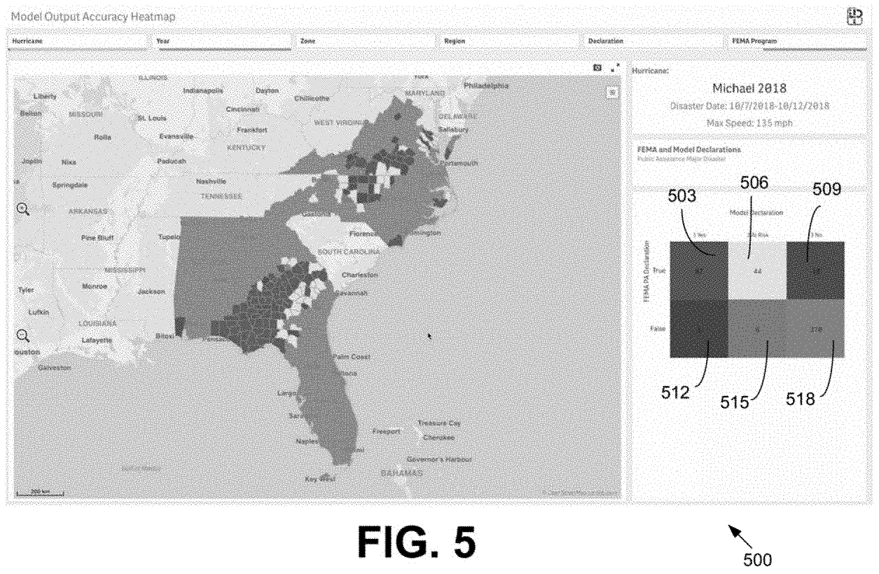

Independent of the visualization, using probabilistic and predictive modeling techniques, a model can be built to project counties likely to be declared disaster areas. Once the model has been refined, it can be back tested on historical hurricanes where the answer was “known” to see how it would perform. These model results are then also visualized in the analytic mode. In the user interface 500 of FIG. 5, different shades 503, 506, 509, 512, 515, 518 are showing true-positives, true-negatives, false-positives, and false-negatives. The counties shaded 503 and 518 are counties that the model accurately predicted, which is the vast majority—in the case of shade 518, the county was predicted as no declaration and FEMA did not declare the county, in the case of shade 503, the county was predicted as yes declaration and FEMA did declare the county. The counties shaded 512 and 509 are the counties that the model inaccurately predicted (false positives and false negatives)—in the case of the shade 509, the county was predicted as a no declaration, but FEMA did declare the county, while in the case of the shade 512, the county was predicted as a yes declaration, yet FEMA did not declare the county. The counties having shades 506 or 515 are the counties our model evaluated as “at risk”—these counties are the in-between counties, where an automatic prediction is not possible with high confidence.

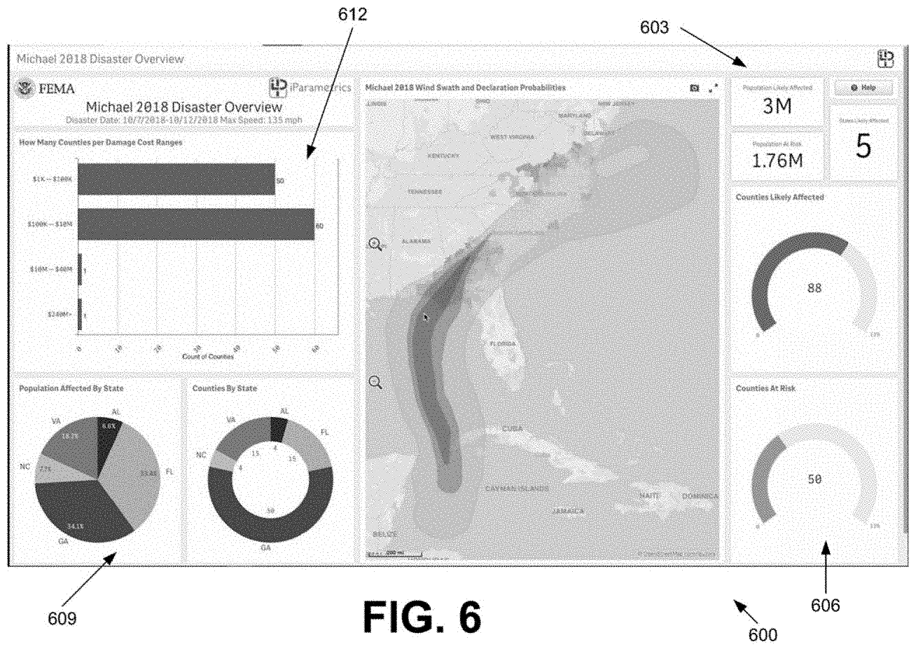

Moving to the operational mode, an example of one instance of the overview dashboard user interface 600 is shown in FIG. 6. This dashboard is targeted to high level executives and leadership. By opening the application, the user can immediately visualize the hurricane wind swath with respect to the counties likely affected and at risk based on the machine learning model. The dashboard also provides the user with major key performance indicators (KPIs) including the number of counties and population likely affected and at risk and breaks down these numbers by state. The dashboard also breaks down the counties by damage cost range.

For example, the panels 603 show that 5 states and 3 million people are affected, with almost an additional 2 million people at risk. The panels 606 show that within these 5 affected states, 88 counties are likely affected with an additional 50 at risk. The breakdown of these counties by state is shown on the panels 609 on the user interface 600 relative to the breakdown of population by state—while GA has the highest number of counties affected, FL had the highest percentage of population affected, showing the user that the counties in FL are more populated than those in GA. Finally, the panel 612 shows a breakdown of counties affected based on damage cost range.

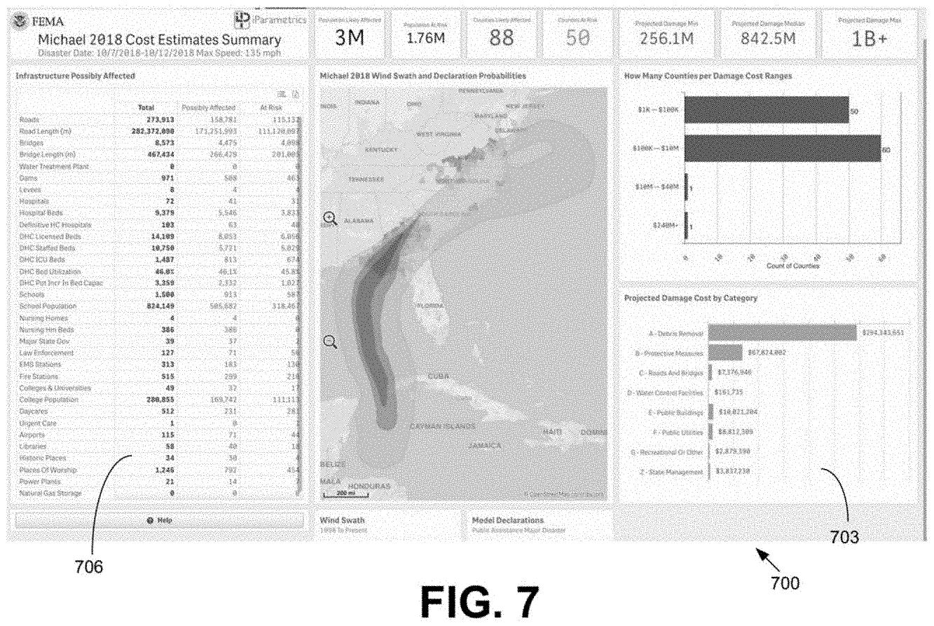

FIG. 7 shows a user interface 700 providing more insight into the projected damage on an event level. This user interface 700 breaks down the high-level statistics more. The user now sees some of the same information from the previous user interfaces, but with addition of projected damage costs by FEMA category as well as the infrastructure, such as roads, bridges, schools, and hospitals, potentially affected by the hurricane event. When FEMA allocates money through the Public Assistance program, they provide funding through eight possible categories, such as Debris Removal, Roads and Bridges, and Public Buildings, so the breakdown of damage by categories as shown at 703 can provide the user with insight into where the majority of damage occurred and what actions need to be taken accordingly.

For example, knowing the anticipated Debris Removal damage can provide guidance with respect to the number of trucks needed to clear the debris. Further, the key infrastructure metrics 706 can give the user the characteristics of the areas affected so that proper resources can be mobilized quickly; in this case, the example of FIG. 7 shows that over 800 thousand school children are potentially affected—knowing this statistic, one can start to answer questions like, “how many temporary school facilities will be needed?” and “how many schools do we need to find that can temporarily serve the affected population?” All of the information can be acquired on an event level, state level, and/or county level.

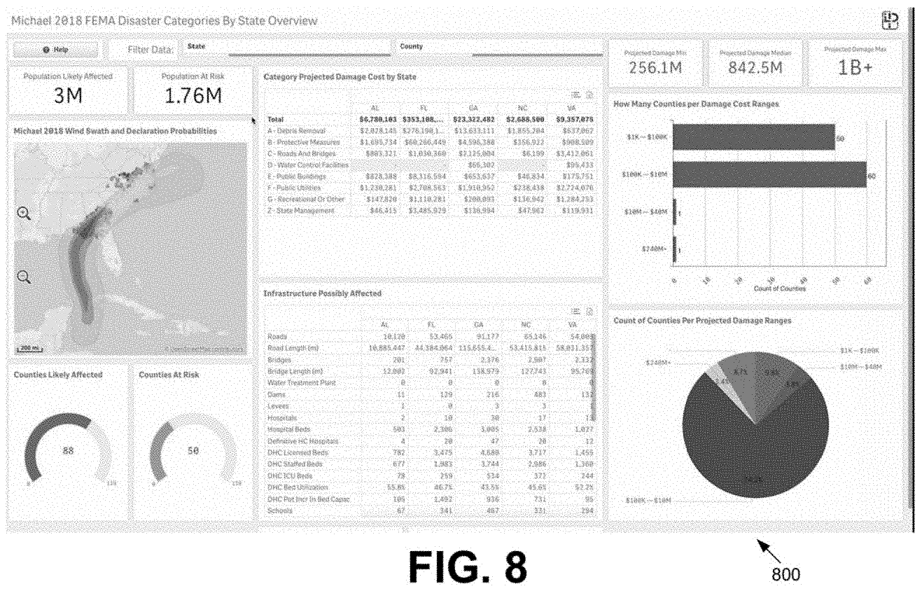

Other user interfaces were developed with state and local stakeholders in mind. These sheets may have more functionality and user interaction enabled than the previous user interface to allow users to not only get an overview, but to also drill-down to a level of detail necessary to be actionable. Similar to the previous sheets, the user can first get an overview as shown in the example user interface 800 of FIG. 8, but this time on a state or regional level as a state or regional manager would not be operationally as interested in other regions.

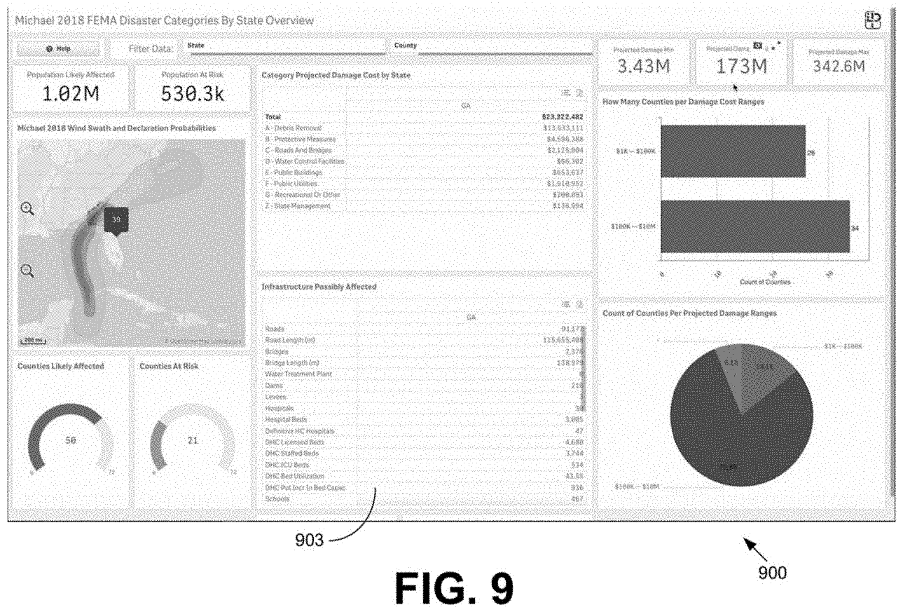

FIG. 9 shows a dashboard user interface 900 customized for a state, e.g., Georgia. For example, the user can filter to Georgia and first see an overview of the disaster in Georgia. On the left, the user interface 900 shows that over 1 million people within 50 counties were likely affected in Georgia with an additional 500 thousand within 21 counties at risk. Further, the GA state manager can expect about 173 million dollars of damage within the state as seen on the right. At 903, one can see the infrastructure possibly affected within Georgia, allowing the user to answer questions like, “how many geotechnical engineers do I need to recruit to respond to the 216 dams possibly affected in GA?” or “where can I relocate the 3 thousand hospital beds affected by this disaster?” While the Overview Dashboard user interface stopped at this level of detail, the State and Local Dashboards provide the user with access to more detailed data.

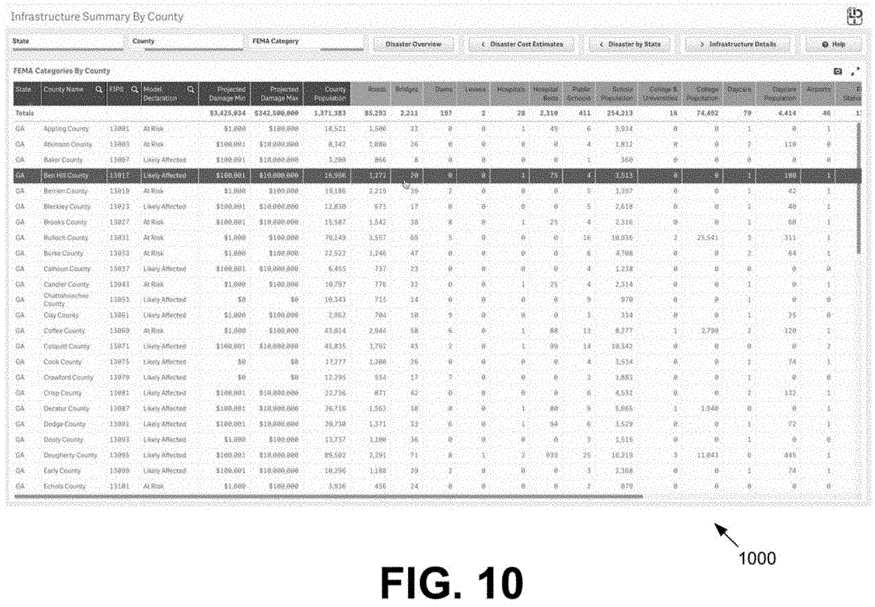

FIG. 10 shows a user interface 1000 with the breakdown of infrastructure not only by state, but also by county. Further, as this tool can be targeted at the Public Assistance process in FEMA, the data is color coded by FEMA category the infrastructure within the table can be filtered by FEMA category. For example, if the user is overseeing the Roads and Bridges response, he or she can filter to “C—Roads and Bridges.” In order to be especially useful for field coordination and personnel, detailed information can be provided regarding each infrastructure type. For example, if the user is in charge of coordinating the response to bridges in Burke County, the user can click on the Bridges cell to be directed to a detailed report.

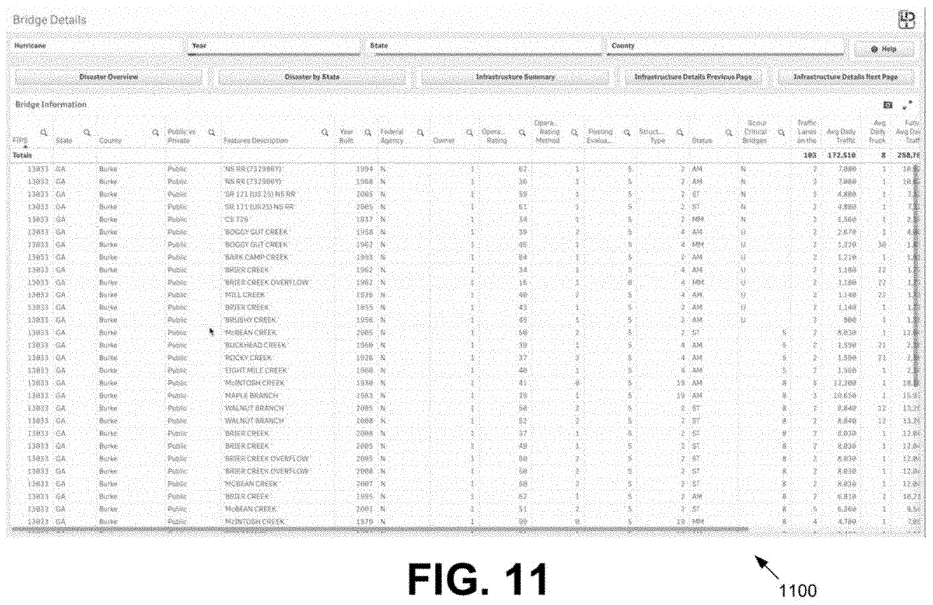

FIG. 11 shows a bridges report user interface 1100 that provides useful information for the coordinator, such as the geographic coordinates and average daily traffic, to allow the coordinator to position personnel efficiently as well as prioritize the response efforts. Based on the report, the coordinator may choose to send personnel to the bridges on McIntosh Creek and Maple Branch over Brier Creek and Mill Creek as the average daily traffic on McIntosh Creek and Maple Branch is about 10 times higher than Brier Creek and Mill Creek. Instead of average daily traffic, the coordinator could also use the Scour Critical Bridges field to prioritize bridge inspection; scour is the removal of streambed material from around bridge abutments or piers and is caused by moving water—as scour deepens, the integrity and stability of a bridge structure can be compromised. As the lower the score is, the more critical the scour, the coordinator may choose to expedite the inspection of the four bridges with the lowest scores.

The user can also export the report (e.g., a spreadsheet, PDF, image) to give to field personnel as the reports also provide useful information for the field personnel. Data fields such as year built, structure type, and operating rating could provide a structural engineer with the background overview needed to know what they can expect when they go out to inspect the bridge.

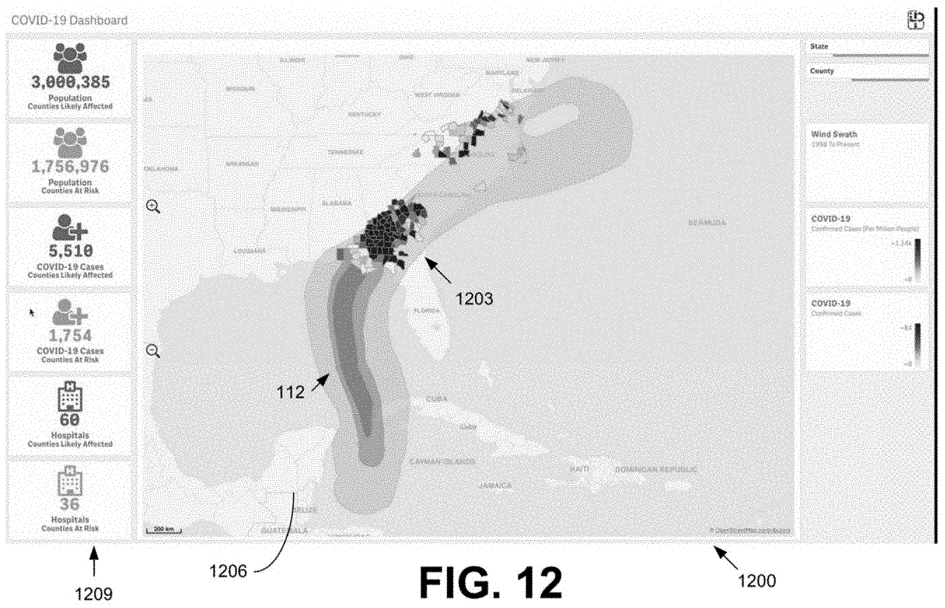

FIG. 12 shows a user interface 1200 with a hypothetical overlap of a hurricane wind swath 112 (like Hurricane Michael) over areas 1203 affected by the COVID-19 pandemic. In FIG. 12, the user can start to understand what the overlap between the two disasters looks like and what the current severity of the disasters is. Specifically, with the map 1206, the user can see the counties likely affected and at risk from the hurricane event shaded according by the number of confirmed COVID-19 cases and relative to the hurricane wind swath. The user can also change the shading of the counties to see the number of confirmed COVID-19 cases per million people. At 1209, the user can see relevant KPIs, such as the population, the number of confirmed COVID-19 cases and the number of hospitals within the likely affected and at-risk counties.

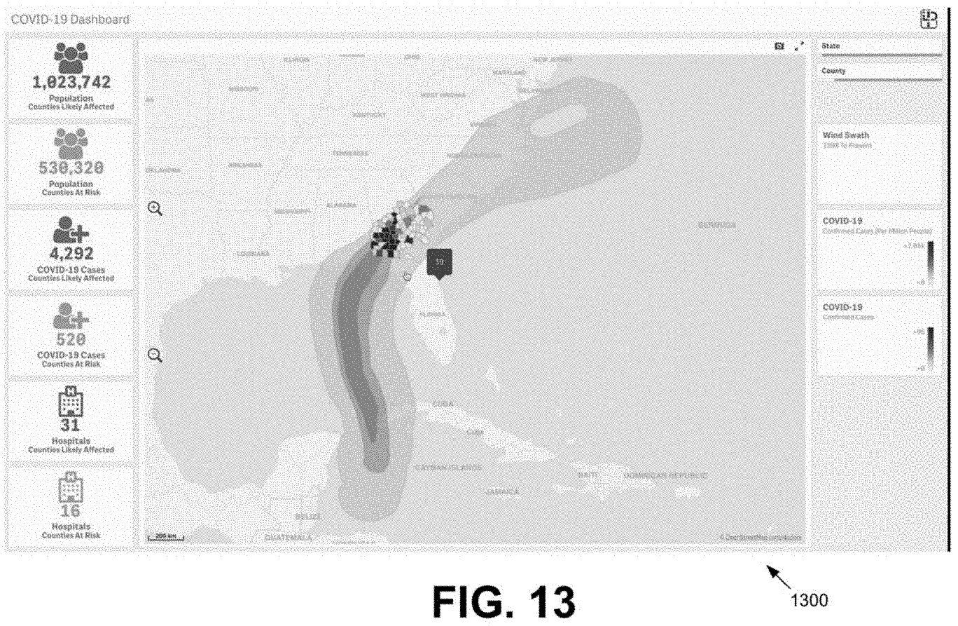

Like the rest of the application, this dashboard user interface 1200 allows for different views based on the user's intended use. FIG. 13 shows a user interface 1300 with a state-specific view for the state of Georgia, where, for example, as of a particular date, there are over 3,800 COVID-19 cases in counties likely affected and 373 cases in counties at risk as well as 32 and 15 hospitals in counties likely affected and at risk.

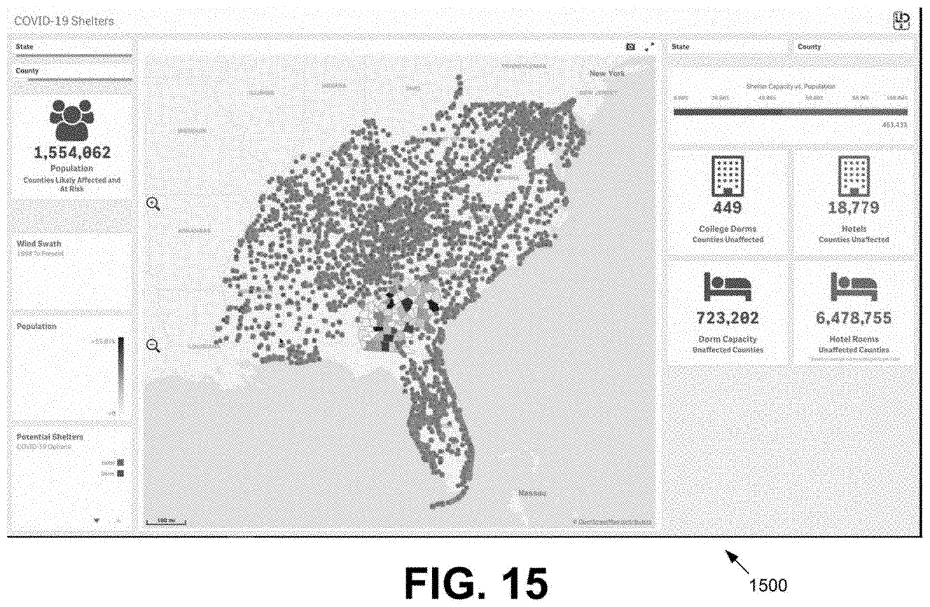

With a hurricane, it is imperative that people likely affected and at risk evacuate their homes, but with the coronavirus, social distancing cannot be disregarded and typical resources, like large evacuation shelters are no longer a safe option. FIG. 14 has a user interface 1400 that helps the user identify safer shelter options in areas predicted to be unaffected by the hurricane. Safe options, such as hotels and college dorms, would allow for social distancing and are probably vacant because of the virus. The user can see the total population that will need to be evacuated on the left and the available shelters and capacities on the right.

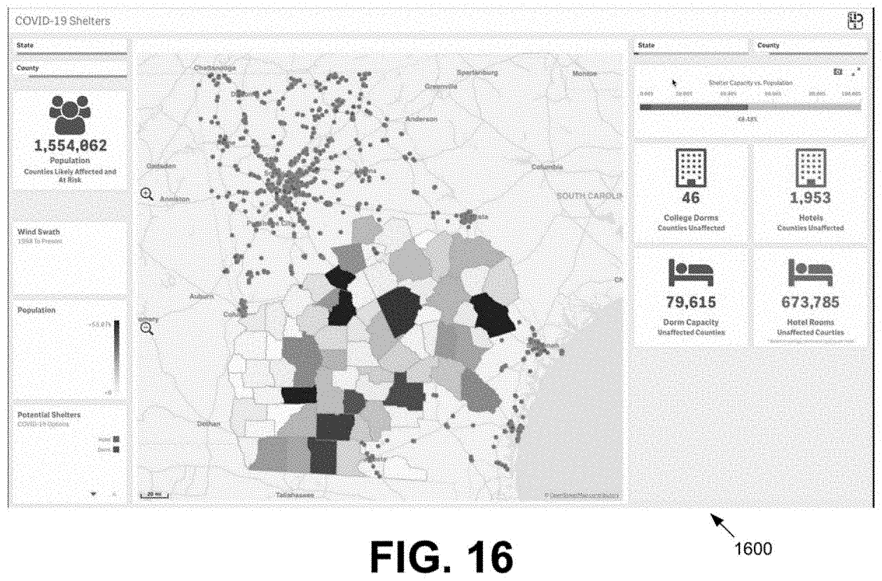

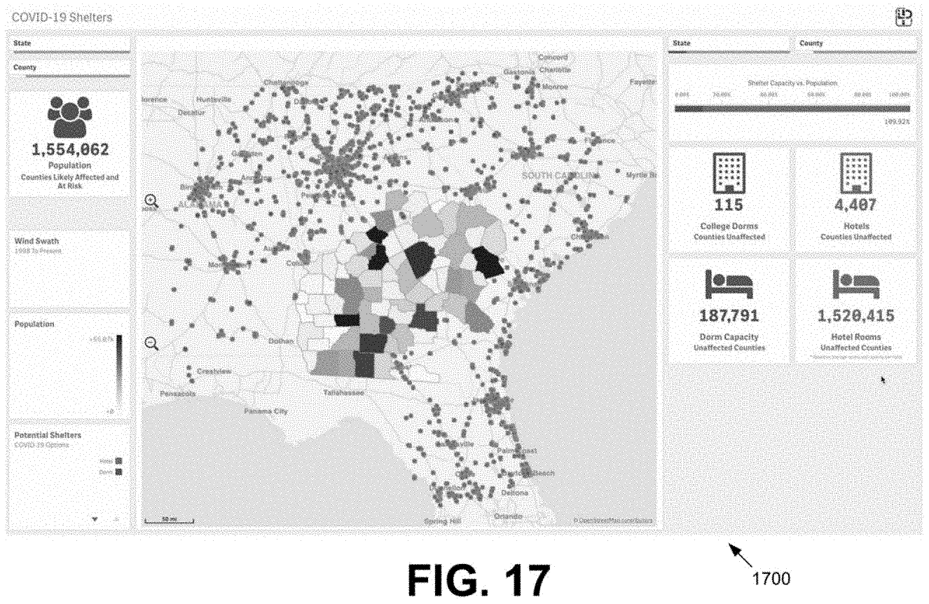

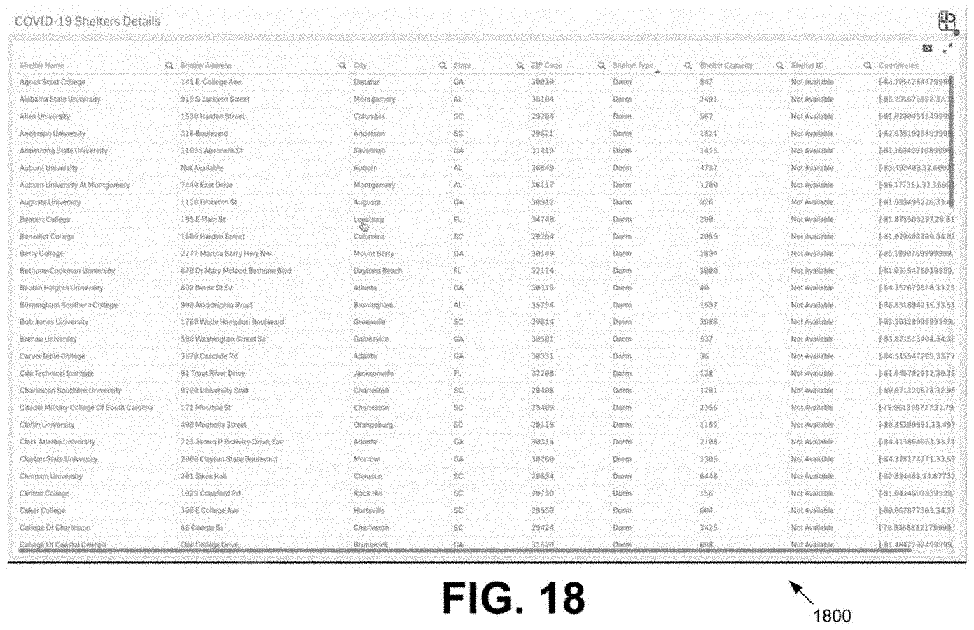

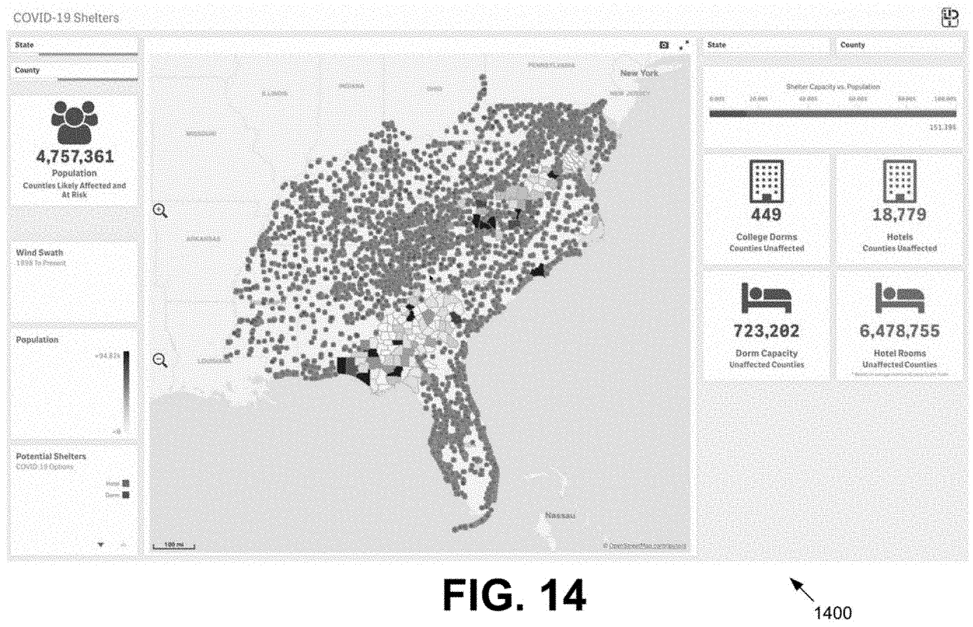

FIG. 15 shows a user interface 1500 with the application of a filter for the state of Georgia and shows that over 1.5 million people may be in need of a shelter—on the right shows there if all shelters in the surrounding states are included, there is more than enough capacity—for example in hotel rooms alone, there is almost three times the needed capacity. But, it also may not be very practical to have a resident from South Georgia evacuate to a shelter in Virginia, so the shelter options can be further filtered as shown on the user interface 1600 in FIG. 16—if the search is restricted to just Georgia shelters, only about 50% of the potential population can be sheltered. The search can be expanded to include Alabama, Florida, and South Carolina and lasso around the closest shelters within 250 miles, yielding the results in the user interface 1700 of FIG. 17. Detailed information about the shelters is shown in the user interface 1800 of FIG. 18, including, names, addresses, types, and capacities.

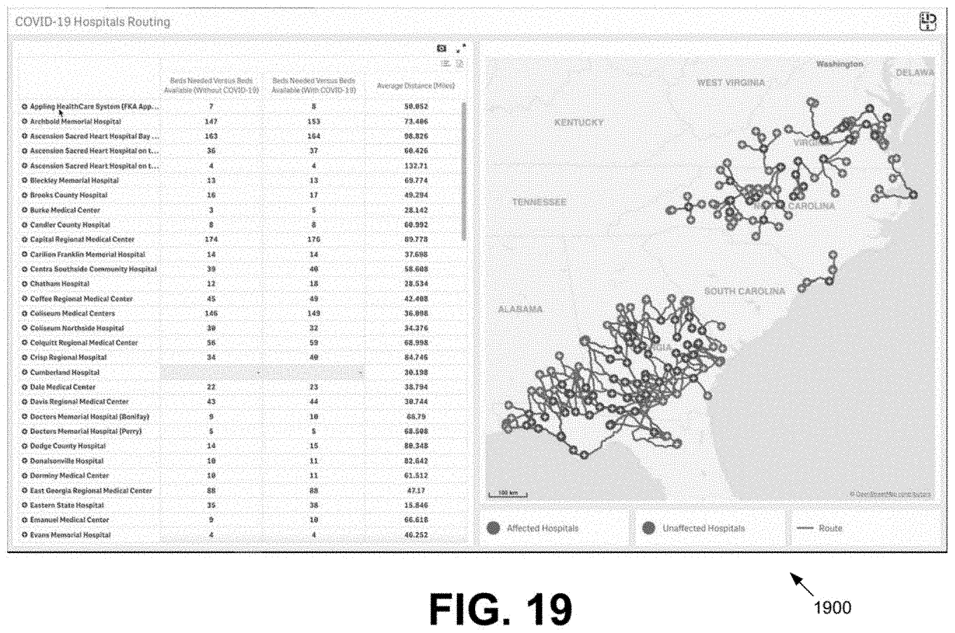

Another complexity deals with the healthcare system and hospitals. If a hurricane were to approach the United States today, response planners would need to quickly coordinate the relocation of hospital patients both with and without the COVID-19 virus among an already stressed healthcare system with a lack of sufficient resources—a logistical nightmare. FIG. 19 shows a user interface 1900 that provides significant assistance in the logistical coordination by providing the user not only with a list of affected hospitals, but also with a list of nearby unaffected hospitals.

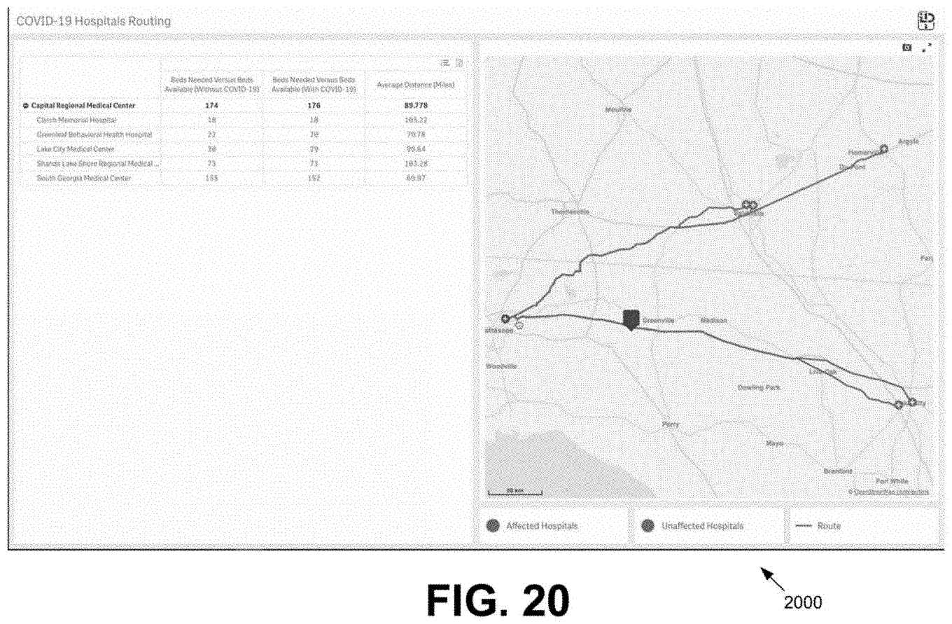

Specifically, the user interface 1900 shows the affected hospitals in bold as well as the number of beds within those hospitals that need to be relocated and distinguishes between counts with the COVID-19 patients and without. For each affected hospital, the sheet also provides the user with the five closest unaffected hospitals as well as the number of beds available within those hospitals and the distance to the hospital. For example, Capital Regional Medical Center has 176 beds that need to be relocated—152 of these beds can be relocated to South Georgia Medical Center, which is the closest unaffected hospital at 70 miles away while the remaining 29 beds could be relocated to the next closest hospitals, South Georgia Medical Center at Lanier and Lake City Medical Center. By selecting Capital Regional Medical Center, the user can also see the hospitals on the map user interface 2000 in FIG. 20 as well as the routing information to get from Capital Regional to the unaffected hospitals.

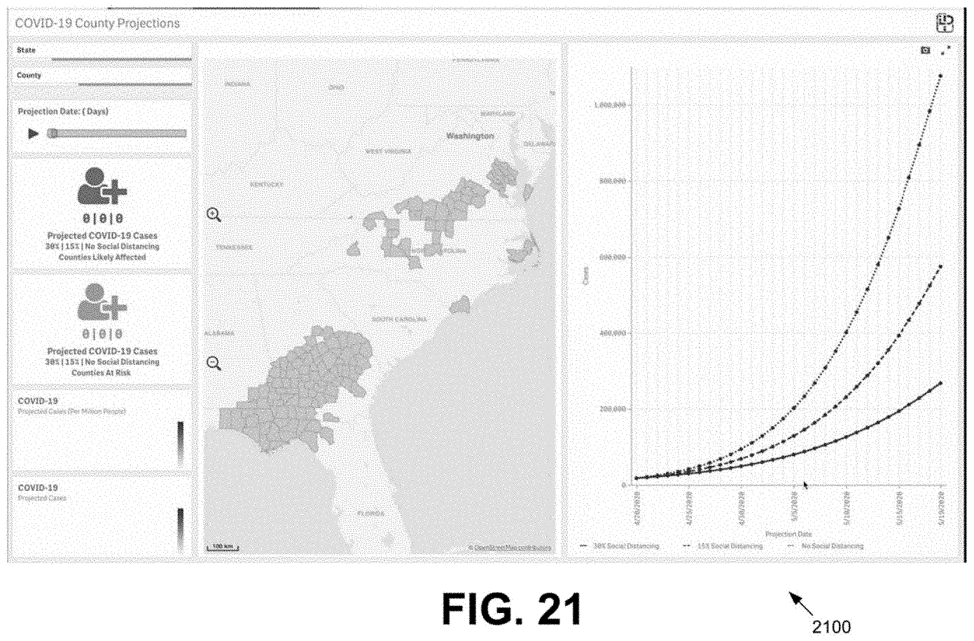

The user interfaces so far have dealt with the “where” issues surrounding evacuations, shelters and hospital relocations, but another important factor is the “when” issue. Public officials have had difficulties with deciding when is the right time to mandate evacuations in the case of hurricanes, and the same difficulties have surfaced in determining when shelter-in-place orders should be mandated and what those orders should look like. FIG. 21 shows a user interface 2100 aiming to assist in making these decisions by allowing officials to ask multiple “what-ifs.”

FIG. 21 provides COVID-19 projections within the context of a hurricane event. The line graph on the right first gives an overall picture of the COVID-19 infections in the next 30 days and under different social distancing measures. A day-by-day view of the projections can be seen by sliding the bar in the top left and looking at the geographic view in the middle and the KPIs on the right. This user interface may be particularly useful when trying to decide on an evacuation date.

For example, an official from Georgia can see how many cases of COVID-19 infections are projected in the next week. Further, the official can see how large of an impact social distancing might have on infections to better determine how stringent social distancing orders should be during the evacuation. From this information, the official could see that if he or she mandated an evacuation today with a 50% reduction in social distancing, the number of COVID-19 infections in a week would be about 4,000 cases higher than projected with the current social distancing in place. As a result, the official might decide that it would be a better decision to push the evacuation back a couple of days to allow people time to plan but maintain the current social distancing requirements.

In the following discussion, a general description of the system and its components is provided, followed by a discussion of the operation of the same.

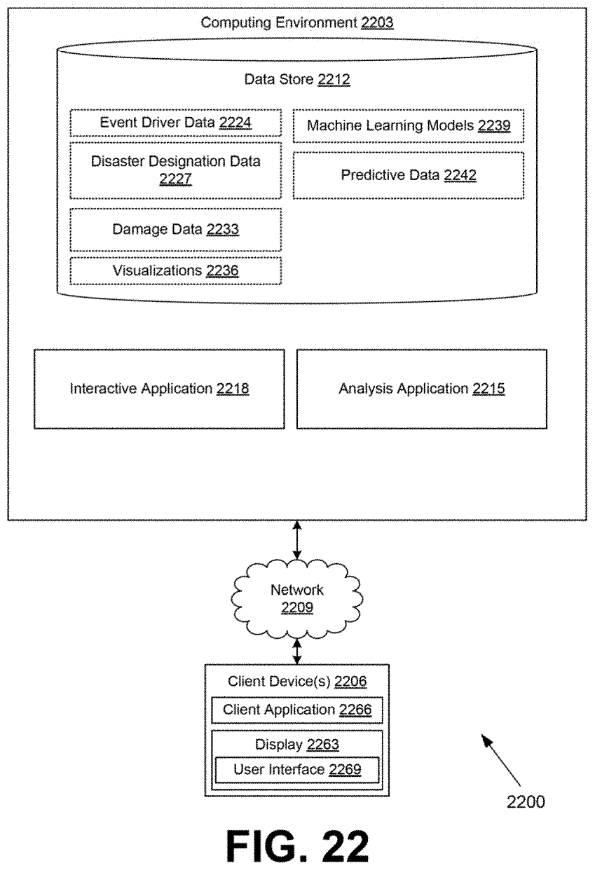

With reference to FIG. 22, shown is a networked environment 2200 according to various embodiments. The networked environment 2200 includes a computing environment 2203 and one or more client devices 2206, which are in data communication with each other via a network 2209. The network 2209 includes, for example, the Internet, intranets, extranets, wide area networks (WANs), local area networks (LANs), wired networks, wireless networks, cable networks, satellite networks, or other suitable networks, etc., or any combination of two or more such networks.

The computing environment 2203 may comprise, for example, a server computer or any other system providing computing capability. Alternatively, the computing environment 2203 may employ a plurality of computing devices that may be arranged, for example, in one or more server banks or computer banks or other arrangements. Such computing devices may be located in a single installation or may be distributed among many different geographical locations. For example, the computing environment 2203 may include a plurality of computing devices that together may comprise a hosted computing resource, a grid computing resource, and/or any other distributed computing arrangement. In some cases, the computing environment 2203 may correspond to an elastic computing resource where the allotted capacity of processing, network, storage, or other computing-related resources may vary over time.

Various applications and/or other functionality may be executed in the computing environment 2203 according to various embodiments. Also, various data is stored in a data store 2212 that is accessible to the computing environment 2203. The data store 2212 may be representative of a plurality of data stores 2212 as can be appreciated. The data stored in the data store 2212, for example, is associated with the operation of the various applications and/or functional entities described below.

The components executed on the computing environment 2203, for example, include an analysis application 2215, an interactive application 2218, and other applications, services, processes, systems, engines, or functionality not discussed in detail herein. The analysis application 2215 is executed to perform analyses on data related to disasters to determine which counties are likely to be declared disaster areas. The interactive application 2218 is executed to generate and update user interfaces that present the analysis and allow for selection and comparison of disaster data and overlays.

The data stored in the data store 2212 includes, for example, event driver 2224, disaster designation data 2227, damage data 2233, one or more visualizations 2236, one or more machine learning models 2239, predictive data 2242, and potentially other data.

The event driver data 2224 includes data relating to weather drivers and static drivers of disaster related events. As discussed above, weather drivers may include, for example, wind swath, precipitation, hail, etc., while static drivers may include population, vegetative cover, etc. The disaster declaration data 2227 indicates which states, counties, or other geographic areas have been designated as disaster areas, which may include designations at the federal level, state level, and/or local level.

The damage data 2233 includes data that inventories damages relating to a disaster event in a given area. The visualizations 2236 correspond to tables, graphs, charts, and/or other graphical elements that can visually represent the event driver data 2224, disaster designation data 2227, and damage data 2233. In some cases, multiple disaster events may be compared and overlaid in the visualizations 2236 (e.g., to view the impact of a hurricane on pandemic-ravaged counties). The visualizations 2236 may be generated at various levels, such as state and county levels.

The machine learning models 2239 are used by the analysis application 2215 in order to perform various analyses on the event driver data 2224 and the disaster declaration data 2227. In particular, the machine learning models 2239 may be trained on historical disaster declaration data in order to generate predictive data 2242, providing predictions whether a given event will result in disaster declarations in specific areas.

The client device 2206 is representative of a plurality of client devices that may be coupled to the network 2209. The client device 2206 may comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, smartwatches, head mounted displays, voice interface devices, or other devices. The client device 2206 may include a display 2263. The display 2263 may comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc.

The client device 2206 may be configured to execute various applications such as a client application 2266 and/or other applications. The client application 2266 may be executed in a client device 2206, for example, to access network content served up by the computing environment 2203 and/or other servers, thereby rendering a user interface 2269 on the display 2263. To this end, the client application 2266 may comprise, for example, a browser, a dedicated application, etc., and the user interface 2269 may comprise a network page, an application screen, etc. The client device 2206 may be configured to execute applications beyond the client application 2266 such as, for example, email applications, social networking applications, word processors, spreadsheets, and/or other applications.

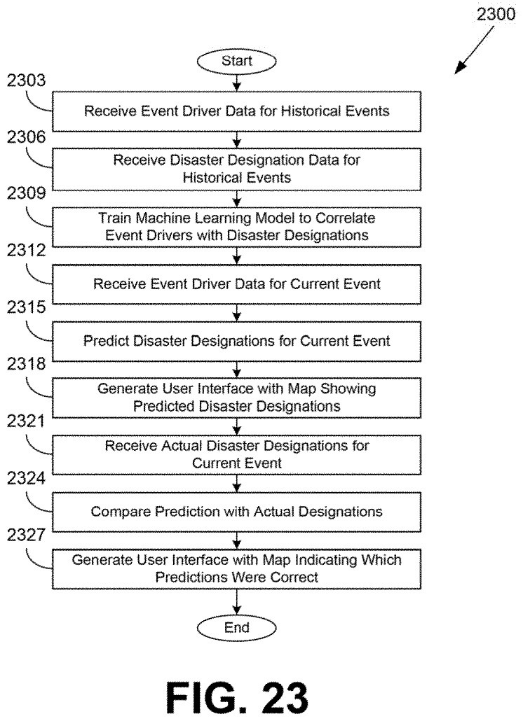

Referring next to FIG. 23, shown is a flowchart 2300 that provides one example of the operation of a portion of the interactive application 2218 (FIG. 22) and/or the analysis application 2215 (FIG. 22) according to various embodiments. It is understood that the flowchart of FIG. 23 provides merely an example of the many different types of functional arrangements that may be employed to implement the operation of the portion of the interactive application 2218 or the analysis application 2215 as described herein. As an alternative, the flowchart of FIG. 23 may be viewed as depicting an example of elements of a method implemented in the computing environment 2203 (FIG. 22) according to one or more embodiments.

Beginning with box 2303, event driver data 2224 is received for one or more historical events. The event driver data may include a plurality of event drivers (including weather drivers and/or static drivers) for a first plurality of geographic areas, such as counties or states. In box 2306, disaster designation data 2227 is received for the historical events, which indicates whether individual ones of the plurality of geographic areas were assigned disaster designations for the historical events. For example, it may be determined whether a county was declared a federal disaster area or a state disaster area.

In box 2309, a machine learning model 2239 is trained to correlate the event drivers with disaster declarations. For example, regression analysis may be used with a random forest model, a K-nearest-neighbors model, a support vector machine, or another machine learning technique in order to discover which event drivers value ranges (e.g., wind, precipitation, population, vegetation cover, etc.) are likely to result in disaster designations.

In box 2312, event driver data 2224 is received for a current event. For example, data for a plurality of event drivers may be received for a second plurality of geographic areas for a current event. In some cases, the first and second pluralities of geographic areas may be identical or may overlap. In box 2315, disaster designations are predicted for the current event based at least in part on the machine learning model 2239. In other words, the machine learning model 2239 predicts which of the geographic areas will be assigned disaster designations for the current event.

In box 2318, a user interface is generated with a map showing the predicted disaster designations. The map visually indicates which of the geographic areas are predicted to be assigned the disaster designation. The indication may be way of a color, shading, pattern, or another technique.

In box 2321, the actual disaster designations are received for the current event, which indicate which of the geographic areas were actually designated to be disaster areas. In box 2324, the predicted disaster designations are compared with the actual disaster designations. In box 2327, a user interface is generated with a map showing which of the predicted disaster designations were correct. The map may indicate which of the predicted disaster designations were incorrect and also which geographic areas were actually designated disaster areas but were not predicted to be assigned the disaster designations. Thereafter, the flowchart 2300 ends.

The system described herein may be implemented in a computing environment including one or more computing devices. Each computing device includes at least one processor circuit, for example, having a processor and a memory, both of which are coupled to a local interface. To this end, each computing device may comprise, for example, at least one server computer or like device. The local interface may comprise, for example, a data bus with an accompanying address/control bus or other bus structure as can be appreciated. The system described herein may be embodied as a cloud-based or Internet-based implementation or as a locally executed application.

Stored in the memory are both data and several components that are executable by the processor. In particular, stored in the memory and executable by the processor are the application described herein and potentially other applications. Also stored in the memory may be a data store and other data. In addition, an operating system may be stored in the memory and executable by the processor.

It is understood that there may be other applications that are stored in the memory and are executable by the processor as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory and are executable by the processor. In this respect, the term “executable” means a program file that is in a form that can ultimately be run by the processor. Examples of executable programs may be, for example, a compiled program that can be translated into machine code in a format that can be loaded into a random access portion of the memory and run by the processor, source code that may be expressed in proper format such as object code that is capable of being loaded into a random access portion of the memory and executed by the processor, or source code that may be interpreted by another executable program to generate instructions in a random access portion of the memory to be executed by the processor, etc. An executable program may be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor may represent multiple processors and/or multiple processor cores and the memory may represent multiple memories that operate in parallel processing circuits, respectively. In such a case, the local interface may be an appropriate network that facilitates communication between any two of the multiple processors, between any processor and any of the memories, or between any two of the memories, etc. The local interface may comprise additional systems designed to coordinate this communication, including, for example, performing load balancing. The processor may be of electrical or of some other available construction.

Although the various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 23 shows the functionality and operation of an implementation of portions of the interactive application 2218 and/or the analysis application 2215. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 403 in a computer system or other system. The machine code may be converted from the source code, etc. If embodied in hardware, each block may represent a circuit or a number of interconnected circuits to implement the specified logical function(s).

Although the flowchart of FIG. 23 shows a specific order of execution, it is understood that the order of execution may differ from that which is depicted. For example, the order of execution of two or more blocks may be scrambled relative to the order shown. Also, two or more blocks shown in succession in FIG. 23 may be executed concurrently or with partial concurrence. Further, in some embodiments, one or more of the blocks shown in FIG. 23 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that comprises software or code can be embodied in any non-transitory computer-readable medium for use by or in connection with an instruction execution system such as, for example, a processor in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device or in multiple computing devices in the same computing environment.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A non-transitory computer-readable medium embodying a program executable in at least one computing device, wherein when executed the program causes the at least one computing device to at least:
receive data indicating a plurality of event drivers for a first plurality of geographic areas respecting one or more historical events;
receive data indicating whether individual ones of the first plurality of geographic areas were assigned first disaster designations for the one or more historical events;
train a machine learning model to determine correlations between the plurality of event drivers and the first disaster designations;
receive data indicating the plurality of event drivers for a second plurality of geographic areas for a current event;
predict which of the second plurality of geographic areas will be assigned second disaster designations for the current event based at least in part on the machine learning model; and
generate a user interface including a map of the second plurality of geographic areas and visually indicating which of the second plurality of geographic areas are predicted to be assigned the second disaster designations.

2. The non-transitory computer-readable medium of claim 1, wherein when executed the program further causes the at least one computing device to at least:
receive data indicating which of the second plurality of geographic areas were actually assigned the second disaster designations;
determine which of the second plurality of geographic areas that were predicted to be assigned the second disaster designations were actually assigned the second disaster designations; and
generate a second user including a second map of the second plurality of geographic areas and visually indicating which of the second plurality of geographic areas were correctly predicted to be assigned the second disaster designations according to the machine learning model.

3. The non-transitory computer-readable medium of claim 2, wherein the second map further visually indicates which of the second plurality of geographic areas were incorrectly predicted to be assigned the second disaster designations according to the machine learning model.

4. The non-transitory computer-readable medium of claim 2, wherein the second map further visually indicates which of the second plurality of geographic areas were actually assigned the second disaster designations but not predicted to be assigned the second disaster designations according to the machine learning model.

5. The non-transitory computer-readable medium of claim 1, wherein the map further includes an overlay of a wind swath from the current event over the second plurality of geographic areas.

6. The non-transitory computer-readable medium of claim 1, wherein the user interface includes one or more panels showing key performance indicators (KPIs) associated with the current event.

7. A system, comprising:
at least one computing device; and
instructions executable in the at least one computing device, wherein when executed the instructions cause the at least one computing device to at least:
receive data indicating a plurality of event drivers for a first plurality of geographic areas respecting one or more historical events;
receive data indicating whether individual ones of the first plurality of geographic areas were assigned first disaster designations for the one or more historical events;
train a machine learning model to determine correlations between the plurality of event drivers and the first disaster designations;
receive data indicating the plurality of event drivers for a second plurality of geographic areas for a current event;
predict which of the second plurality of geographic areas will be assigned second disaster designations for the current event based at least in part on the machine learning model; and
generate a user interface including a map of the second plurality of geographic areas and visually indicating which of the second plurality of geographic areas are predicted to be assigned the second disaster designations.

8. The system of claim 7, wherein when executed the instructions further cause the at least one computing device to at least:
receive data indicating which of the second plurality of geographic areas were actually assigned the second disaster designations;
determine which of the second plurality of geographic areas that were predicted to be assigned the second disaster designations were actually assigned the second disaster designations; and
generate a second user including a second map of the second plurality of geographic areas and visually indicating which of the second plurality of geographic areas were correctly predicted to be assigned the second disaster designations according to the machine learning model.

9. The system of claim 8, wherein the second map further visually indicates which of the second plurality of geographic areas were incorrectly predicted to be assigned the second disaster designations according to the machine learning model; and
wherein the second map further visually indicates which of the second plurality of geographic areas were actually assigned the second disaster designations but not predicted to be assigned the second disaster designations according to the machine learning model.

10. The system of claim 7, wherein the user interface includes one or more components that when selected causes the map to be updated to show one or more static drivers associated with the current event in the second plurality of geographic areas.

11. The system of claim 7, wherein the user interface includes one or more components that when selected causes the map to be updated to show one or more weather drivers associated with the current event in the second plurality of geographic areas.

12. A method, comprising:
receiving, by at least one computing device, data indicating a plurality of event drivers for a first plurality of geographic areas respecting one or more historical events;
receiving, by the at least one computing device, data indicating whether individual ones of the first plurality of geographic areas were assigned first disaster designations for the one or more historical events;
training, by the at least one computing device, a machine learning model to determine correlations between the plurality of event drivers and the first disaster designations;
receiving, by the at least one computing device, data indicating the plurality of event drivers for a second plurality of geographic areas for a current event;
predicting, by the at least one computing device, which of the second plurality of geographic areas will be assigned second disaster designations for the current event based at least in part on the machine learning model; and
generating, by the at least one computing device, a user interface including a map of the second plurality of geographic areas and visually indicating which of the second plurality of geographic areas are predicted to be assigned the second disaster designations.

13. The method of claim 12, further comprising:
receiving, by the at least one computing device, data indicating which of the second plurality of geographic areas were actually assigned the second disaster designations;
determining, by the at least one computing device, which of the second plurality of geographic areas that were predicted to be assigned the second disaster designations were actually assigned the second disaster designations; and
generating, by the at least one computing device, a second user including a second map of the second plurality of geographic areas and visually indicating which of the second plurality of geographic areas were correctly predicted to be assigned the second disaster designations according to the machine learning model.

14. The method of claim 13, wherein the second map further visually indicates which of the second plurality of geographic areas were incorrectly predicted to be assigned the second disaster designations according to the machine learning model.

15. The method of claim 13, wherein the second map further visually indicates which of the second plurality of geographic areas were actually assigned the second disaster designations but not predicted to be assigned the second disaster designations according to the machine learning model.

16. The method of claim 12, wherein the map further includes an overlay of a wind swath from the current event over the second plurality of geographic areas.

17. The method of claim 12, wherein the user interface includes one or more panels showing key performance indicators (KPIs) associated with the current event.

18. The method of claim 12, wherein the user interface includes one or more components that when selected causes the map to be updated to show one or more static drivers associated with the current event in the second plurality of geographic areas.

19. The method of claim 12, wherein the user interface includes one or more components that when selected causes the map to be updated to show one or more weather drivers associated with the current event in the second plurality of geographic areas.

20. The method of claim 12, wherein the user interface includes one or more components that when selected causes the map to be updated to show one or more weather drivers associated with the current event along with an impact of another current event in the second plurality of geographic areas.
